(19) United States
(12) Patent Application Publication
(10) Pub. No.: US 2023/0405345 A1
(43) Pub. Date: Dec. 21, 2023

(54) Implantable Neurophysiology Devices

(71) Applicant: BrainConnect Pty Ltd, Marrickvile (AU)

(72) Inventors: Omid Kavehei, Marrickville (AU); Arman Ahnood, Lalor (AU); Nhan Duy Truong, Hinchinbrook (AU)

(73) Assignee: BrainConnect Pty Ltd, Marrickvile (AU)

(21) Appl. No.: 18/032,142

(22) PCT Filed: Oct. 29, 2021
(86) PCT No.: PCT/IB2021/060057

§ 371 Date: Apr. 14, 2023

Related U.S. Application Data
(60) Provisional application No. 63/127,455, filed on Dec. 18, 2020.

(51) Int. Cl. A61N 1/378; A61N 1/36; A61N 1/372; A61N 1/05

(52) U.S. Cl. CPC A61N 1/3787; A61N 1/0529; A61N 1/37247; A61N 1/3605

(57) ABSTRACT

An implantable device has a slim carrier with first and second sides. The two sides each have a signal electrode and a body potential electrode. The body potential electrodes are internally connected. Electrodes on opposing sides are aligned. An insulating extension of insulating material extends beyond a perimeter of the carrier to increase device sensitivity. If the carrier is hollow, there may be an IC inside to provide active functions including power management, communication, device control, and signal storage. The IC may include an amplifier and an ADC to sense signals, that it may store in memory and/or communicate to an external interface unit (EIU). The IC may include a DAC and a power amplifier to electrically stimulate tissue with signals received from the EIU.

### CROSS REFERENCES TO RELATED APPLICATIONS

This application is a U.S. national stage filing of PCT application serial no. PCT/IB2021/060057, entitled “Implantable Neurophysiology Devices”, filed Oct. 29, 2021, which claims priority from U.S. provisional patent application Ser. No. 63/127,455, entitled “Implantable Neurophysiology Devices”, filed on Dec. 18, 2020. The priority applications are hereby incorporated by reference as if set forth in full in this application for all purposes.

### TECHNICAL FIELD

The disclosed technology relates generally to implantable medical devices, and in particular to those for long-term monitoring or stimulation of brain activity. Implementations may further be used for measuring and/or stimulating electrical activity in any other part of the body.

### BACKGROUND

Unless otherwise indicated herein, elements described in this section are not prior art to the claims and are not admitted being prior art by inclusion in this section.

Brain activity is monitored for a variety of reasons, particularly for diagnosing medical conditions. However, it is usually performed for a short time only, since it may require hooking up a patient to extensive equipment, temporarily connecting electrodes to the scalp, and other inconvenient measures. Long-term monitoring and/or stimulation may require invasive surgery, and electrodes may be placed under the scalp or within the brain. This is the case, for instance, with cochlear implants that help some classes of deaf people to hear, or for deep brain stimulation, such as might be used for patients with Parkinson's or Alzheimer's disease. Because of the invasiveness, these methods have not been widely used for, for example, epileptic patients. Until now, only a few methods have existed to provide less invasive long-term monitoring or stimulation. However, such methods also have come with downsides, including the size of implanted devices, and issues with wiring through the tissues.

Ambulatory and long-term neurophysiology could provide a better understanding of neurological disorders, brain development, and cognitive psychology. When it comes to diseases like epilepsy, long-term brain activity monitoring could hold the key to breakthrough developments such as seizure forecasting and automatic seizure logging systems. A survey of 1,056 epilepsy patients, caregivers, and family members has ranked seizure predictability as the highest priority goal for improving their lives. It is almost a unified belief in the research community that a long-term electroencephalogram (EEG) could hold the key to identifying patient-specific biomarkers for seizure prediction. Life-long, continuous, reliable, minimally invasive, and accurate EEG monitoring could also be the best way to predict and stop seizures, and hence, injuries or sudden unexpected death in epilepsy (SUDEP), which is a risk for all people who are living with epilepsy. Today, such monitoring is not available. Instead, monitoring is paper-based or relies on smart phone apps.

Yet, the industry overall has made progress. For example, U.S. Pat. No. 10,543,372 by Prawer et al. describes a method of forming an enclosure for medical devices using biocompatible materials and suitable for forming hermetically sealed seals. The method employs a diamond material to provide two half enclosure components inside of which electronic circuits can reside. U.S. Pat. No. 10,601,255 by Pigeon et al. describes how a collimated beam can be used to transmit energy and data to a remote device. Such a device may be an implanted medical device, thus obviating the need to replace the device as its battery gets empty or nears end-of-life. Other methods have also been developed, such as energy harvesting based on a patient's motion, and energy provision and data communication through electromagnetic field backscattering, such as in a radio-frequency identification (RFID) system. These methods have been well documented in the art.

Nevertheless, the current state of technology doesn't adequately address several medical issues. Epilepsy diagnosis and prognosis are two such issues. Currently, a patient self-declare or self-report system is in place, which does not provide a reliable way of reporting the number of seizures. Misdiagnosis accounts for financial, physical, and mental hits in terms of mistreatment of the disease. Misdiagnosis in epilepsy happens because, in many cases, seizures are not frequent and may not occur when the patient is under short-term monitoring. Current devices for long-term diagnosis use electrodes with long leads. This gives a reliability problem when leads fracture or become dislodged. Leads implanted in the growing heads of children pose additional challenges.

There is an unmet need for a brain interface that can be implanted easily, minimally invasively, and that can operate for many years without bothering the patient. The brain interface should be capable of sensing signals from and/or providing signals to the body. It could enable automatic seizure detection, automatic seizure logging, and reliable seizure prediction.

### SUMMARY

Interfacing with the brain's electrical activity via a long-term, and potentially life-long, neurotechnology has been the dream and aspiration of many. With no cure in sight for people with serious neurological disorders such as epilepsy, and an increase in severity and prevalence of other conditions such as addiction and severe depression, the importance of enabling long-term monitoring is greater than ever. Implementations of the disclosed technology provide a minimally invasive subgaleal device that enables potentially life-long monitoring using a few, leadless, electrodes. Such monitoring may include prolonged electroencephalogram (EEG) recording, a gold standard for epilepsy diagnosis. Implementations may be powered in a variety of ways, including by wireless power transfer such as capacitive coupling, inductive or electromagnetic coupling (with a magnetic field or radio-frequency (RF) waves), optics, ultrasonics, as well as by energy harvesting methods, such as motion-based or biomechanical energy harvesting.

In a first aspect, an implementation provides an implantable device. It includes a carrier with a first side and a second side. The first side and the second side are on opposite surfaces. The implantable device is slim: the surfaces may be less than 5 mm apart. The device has a first signal electrode with a first area, shape, and orientation, embedded on the first side, and a second signal electrode with a second area, shape, and orientation, embedded on the second side. It has a first body potential electrode with a third area, shape, and orientation, embedded on the first side; and a second body potential electrode with a fourth area, shape, and orientation, embedded on the second side. The second body potential electrode is electrically coupled to the first body potential electrode. The first signal electrode may be aligned with the second signal electrode, and the first body potential electrode may be aligned with the second body potential electrode. An implementation may comprise two or more pairs of lateral-signal electrodes on the second side, located on a circle centered around the second signal electrode. An insulating extension of a biocompatible electrically insulating material may extend beyond a perimeter of the carrier to increase the resistance of a path through tissue between the first signal electrode and the second signal electrode. The insulating extension helps to increase the device's sensitivity for radial signals, or the power it delivers in case of stimulation. On the second side, there may be three or more lateral-signal electrodes, located around the second signal electrode. The carrier may comprise diamond, ceramics, metal, and/or organic material.

The carrier may be hollow, and an integrated circuit (IC) may be mounted inside to provide active functionality. The IC is connected with the four (or more) electrodes and includes a power management system that receives power from an external interface unit (EIU) or harvested power. The power management system is coupled with the body potential electrodes and with a power transducer or harvester. A power transducer may work capacitively using electrodes, electromagnetically using an inductor, or optically using, for example, a photovoltaic cell or other optical power device. The IC may also include a communication interface to communicate with the EIU. The communication interface may work capacitively, electromagnetically, or optically. A power transducer and the communication interface may be combined, or separate. Some implementations include a body referencing circuit (BRC) to provide a voltage to the body potential electrodes. Further implementations include an amplifier and an analog-to-digital converter (ADC) to sense signals in the body. An implementation may further include a sensor, coupled with an amplifier that amplifies sensor signals, and provides it to the ADC. The sensor may be an accelerometer. Further implementations may include a digital-to-analog converter (DAC) and a power amplifier to stimulate the body by applying electrical signals to it.

In a second aspect, an implementation provides an implantable device. It includes a carrier with a first side and a second side. The first side and the second side are on different surfaces, or opposite surfaces. The device includes two electrodes on each the first side and the second side. The device is configurable, allowing to short-circuit two of the electrodes to each other to form a pair of body potential electrodes.

In a third aspect, implementations provide a method of manufacturing an implantable device as described above. The electrodes include a first signal electrode, a second signal electrode, a first body potential electrode, and a second body potential electrode. The first and second body potential electrodes are short-circuited to each other. Some implementations increase insulation between electrodes on the first side and electrodes on the second side by placing a biocompatible electrically insulating material beyond a perimeter of the carrier.

A further understanding of the nature and the advantages of particular implementations disclosed herein may be realized by reference of the remaining portions of the specification and the attached drawings.

### BRIEF DESCRIPTION OF THE DRAWINGS

The disclosed technology will be described with reference to the drawings, in which:

FIG. 1 illustrates a sectional view of a passive implantable device in an implementation of the disclosed technology.

FIG. 2 illustrates a top view of a passive implantable device in an implementation of the disclosed technology.

FIG. 3 illustrates an alternative top view of a passive implantable device in an implementation of the disclosed technology.

FIG. 4 illustrates another alternative top view of a passive implantable device in an implementation of the disclosed technology.

FIGS. 5(a) and (b) each illustrate a top/bottom view 500 of an implementation of the disclosed technology that is configured for measuring and/or compensating tangential signals.

FIG. 5(c) illustrates how the impact of electrode-tissue displacement caused by muscular activity, for example head movement in a sheep study, is reduced with the use of an insulating extension in an implementation of the disclosed technology.

FIGS. 6(a) and (b) illustrate top/bottom views of alternative implementations of the disclosed technology that are configured for measuring and/or compensating tangential signals.

FIG. 7 shows in-vitro measurement results for different radii of the insulating extension in an implementation of the disclosed technology.

FIG. 8 illustrates a sectional view of an example active implantable device with capacitive power transfer and communication in an implementation of the disclosed technology.

FIG. 9 illustrates a sectional view of an example active implantable device with electrodes as shown in FIG. 6.

FIG. 10 illustrates a sectional view of an example active implantable device with inductive or electromagnetic power transfer and communication in an implementation of the disclosed technology.

FIG. 11 illustrates a sectional view of an example active implantable device with optical power transfer and inductive or electromagnetic communication in an implementation of the disclosed technology.

FIG. 12 illustrates a sectional view of an example active implantable device in an implementation of the disclosed technology.

FIG. 13 illustrates a top view of the implantable device of FIG. 12 in an implementation of the disclosed technology.

FIG. 14 illustrates a bottom view of the implantable device of FIG. 12 in an implementation of the disclosed technology.

FIG. 15 illustrates an implantable device with configurable body potential electrodes in an implementation of the disclosed technology.

FIG. 16 illustrates an example functional architecture of an active sensing device in an implementation of the disclosed technology.

FIG. 17 illustrates an example functional architecture of an active sensing device with an extra sensor in an implementation of the disclosed technology.

FIG. 18 illustrates an example functional architecture of an active stimulating device in an implementation of the disclosed technology.

FIG. 19 illustrates an example functional architecture of an active sensing device with extra sensors and both radial and tangential sensing in an implementation of the disclosed technology.

FIG. 20 illustrates an example external system in an implementation of the disclosed technology.

FIG. 21 illustrates a method of manufacturing an implantable device in an implementation of the disclosed technology.

In the figures, like reference numbers may indicate functionally similar elements. The systems and methods illustrated in the figures, and described in the Detailed Description below, may be arranged and designed in a wide variety of different implementations. Neither the figures nor the Detailed Description are intended to limit the scope as claimed. Instead, they merely represent examples of different implementations of the disclosed technology.

### DETAILED DESCRIPTION

General

Interfacing with the brain's electrical activity via a long-term, and potentially life-long, neurotechnology has been the dream and aspiration of many. With no cure in sight for people with serious neurological disorders such as epilepsy, and an increase in severity and prevalence of other conditions such as addiction and severe depression, the importance of enabling long-term monitoring is greater than ever. Implementations of the disclosed technology provide a minimally invasive subgaleal device that enables potentially life-long monitoring using a few, leadless, electrodes. Such monitoring may include prolonged electroencephalogram (EEG) recording, a gold standard for epilepsy diagnosis. Further implementations provide an implantable device that may be implanted anywhere in the body or brain, and/or a device that provides electrical stimulation to the body or brain. Implementations may be powered in a variety of ways, including by wireless power transfer such as capacitive coupling, inductive or electromagnetic coupling (with a magnetic field or radio-frequency (RF) waves), optics, ultrasonics, as well as by energy harvesting methods, such as motion-based or biomechanical energy harvesting.

Implementations of the disclosed technology provide passive and active interfaces to the brain, or to any other part of the body. Passive interfaces (“passive devices”) may require leads to carry signals between a device and the source or destination of data. For example, a passive device may have been subcutaneously implanted, but requires a thin cable exiting through the skin to carry measured data from the device to an external amplifier and recorder. Active interfaces (“active devices”) may be leadless. They can harvest or receive energy from a source outside or inside the body, and use this energy to measure, log, and transmit the data to an external interface unit (EIU), or to receive data from the EIU, process it and electrically stimulate the body in accordance with the received data. The EIU transmits and/or receives and processes the data. The EIU may also deliver the power to the device. In case of a brain interface, a device may be implanted subgaleally, residing between the scalp and the skull. The EIU may sit on the scalp and interface wirelessly through the skin.

Passive Devices

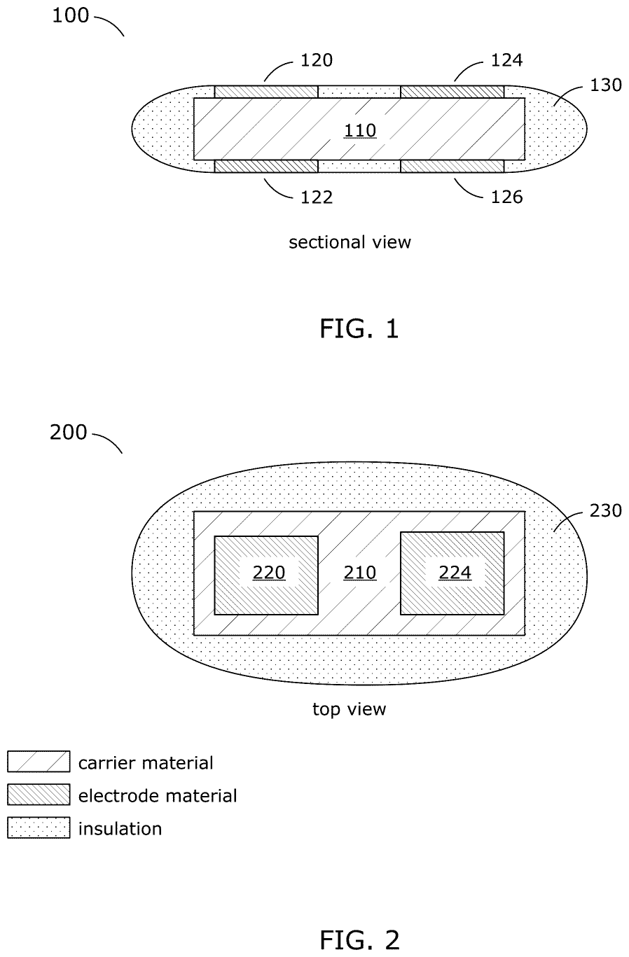

FIG. 1 illustrates a sectional view of a passive implantable device in an implementation of the disclosed technology. A passive device 100 includes a carrier 110, a first signal electrode 120, a second signal electrode 122, a first body potential electrode 124, a second body potential electrode 126, and may include an insulating extension 130 of insulating material beyond the carrier's perimeter.

FIG. 1 shows a sectional view that cuts vertically through passive device 100 (naming of the directions vertical and horizontal, as well as top, bottom, etc. are for convenience of this description only, and devices may be used in any orientation that is medically justified or required). Carrier 110 may be solid or hollow, and it may be made from a single piece of material or from two or more components sealed together. Carrier 110, made of a first biocompatible electrically insulating material, has a first side and a second side, drawn here as its top and its bottom, that may be in parallel planes, or on different (e.g., opposing) surfaces. In some implementations the first side and the second side may not be flat, but curved, or each may occupy multiple planes or surfaces, where parts of the first and the second side are opposing. The first side comprises first signal electrode 120 and first body potential electrode 124, whereas the second side comprises second signal electrode 122 and second body potential electrode 126. Although not drawn in FIG. 1, first body potential electrode 124 is electrically coupled (short-circuited) to second body potential electrode 126. First signal electrode 120 has a first area and first shape in a first orientation. Second signal electrode 122 has a second area and second shape in a second orientation. First body potential electrode 124 has a third area and third shape in a third orientation. Second body potential electrode 126 has a fourth area and fourth shape in a fourth orientation. Insulating extension 130 is made of a second biocompatible electrically insulating material. It exposes first signal electrode 120, second signal electrode 122, first body potential electrode 124, and second body potential electrode 126.

In some implementations, the first side and the second side are less than 5 millimeters (5 mm) apart, or as drawn, passive device 100 is no more than 5 mm thick. In further implementations, first signal electrode 120 is placed in parallel with and aligned with second signal electrode 122, and first body potential electrode 124 is placed in parallel with and aligned with second body potential electrode 126. In these implementations, the first area and the first shape may equal the second area and the second shape, and the third area and the third shape may equal the fourth area and the fourth shape. The first orientation matches the second orientation, and the third orientation matches the fourth orientation. As drawn, second signal electrode 122 is placed directly facing first signal electrode 120, and second body potential electrode 126 is placed directly facing first body potential electrode 124. For electrodes to be aligned, their shape, size, and orientation may be the same, and they may be placed in parallel planes or on opposing surfaces. In curved devices, an electrode on an outer side of the curve may be slightly larger than an electrode on an inner side of the curve.

In implementations, carrier 110 and insulating extension 130 may be made from any electrically insulating materials suited for implantation in the body, including but not limited to carbon, diamonds (monocrystalline, polycrystalline, microcrystalline and nanocrystalline), organics and polymers (such as SU8 or other types of epoxy, polyimide, different types of elastomer, different forms of parylene), and ceramics (aluminum oxide, aluminum nitride, glass, zirconium oxide, etc.). The electrodes (first signal electrode 120, second signal electrode 122, first body potential electrode 124, and second body potential electrode 126) may be made from metal, such as platinum (in any of its forms), tungsten, titanium, gold, or any other metal that is suitable for implanting, or they may be carbon-based, such as diamond (e.g. boron-doped, nitrogen-doped, or otherwise doped, for any form of diamond, including monocrystalline, polycrystalline, microcrystalline and nanocrystalline), graphite, graphene, or any combination of diamond, graphite, and graphene, or they may comprise metal oxides and/or metal nitrites/nitrides (such as titanium nitride, indium tin oxide, iridium oxide), or any other electrically conductive materials suited for implantation.

In an example implementation, passive device 100 includes a polycrystalline diamond (PCD) carrier 110 on which electrodes have been grown by chemical vapor deposition (CVD). The electrodes may be doped, for instance with nitrogen, such that the electrodes comprise n-type ultra-nanocrystalline diamond (N-UNCD). Alternatively, electrodes may have been doped with boron, such that the electrodes comprise p-type ultra-nanocrystalline diamond (P-UNCD). Yet alternatively, electrodes may have any type of doping embedded in any suitable manufacturing process. The insulating extension 130 may be made of, or comprise, polydimethylsiloxane (PDMS).

Since carrier 110 is thin, first signal electrode 120 and second signal electrode 122 are located in very close proximity to each other. Therefore, there exists a conductive path through the body tissues between the two electrodes. Insulating extension 130 increases the length of the conductive path, and therefore increases the electrical resistance measured through the body tissues between the two electrodes. The higher resistance between the electrodes enables a larger differential voltage, which is advantageous for measuring electrical signals in, or alternatively for electrical stimulation of, the adjacent tissue. Similarly, insulating extension 130 increases the electrical resistance of the path through live tissue between the very closely situated first body potential electrode 124 and second body potential electrode 126. However, because first body potential electrode 124 is electrically coupled to second body potential electrode 126, a potential measured on these two electrodes is likely to be close to an average potential measured between first signal electrode 120 and second signal electrode 122. For purpose of measurement or stimulation, this potential can serve as the body potential, or at least as a common-mode potential for any measured electrical activity.

FIG. 1 does not show how passive device 100 is coupled with any measurement equipment or stimulation devices. Such equipment or devices could sit outside of the body, or may be situated elsewhere inside the body. Implementations are coupled via a cable that includes at least three different conductors (one for each signal electrode, and one for the two body potential electrodes). The cable may enter passive device 100, for example, via a hole in insulating extension 130, and connect to the electrodes.

Although FIG. 1 shows passive device 100 with two signal electrodes and two body potential electrodes, some implementations may have more than two signal electrodes and/or more than two body potential electrodes. All such variations are within the scope and ambit of the presently disclosed technology.

FIG. 2 illustrates a top view 200 of a passive implantable device in an implementation of the disclosed technology. The view of this device comprises a carrier 210, a first signal electrode 220, a first body potential electrode 224, and an insulating extension 230. Although insulating extension 230 is shown with an oval shape, implementations may use a round shape, or any other shape that increases the length of a conductive path through body tissues between top and bottom electrodes. The implementation in FIG. 2 shows (the optional) insulating extension 230 surrounding the perimeter of carrier 210, but not covering its top. Similarly, it may not cover its bottom.

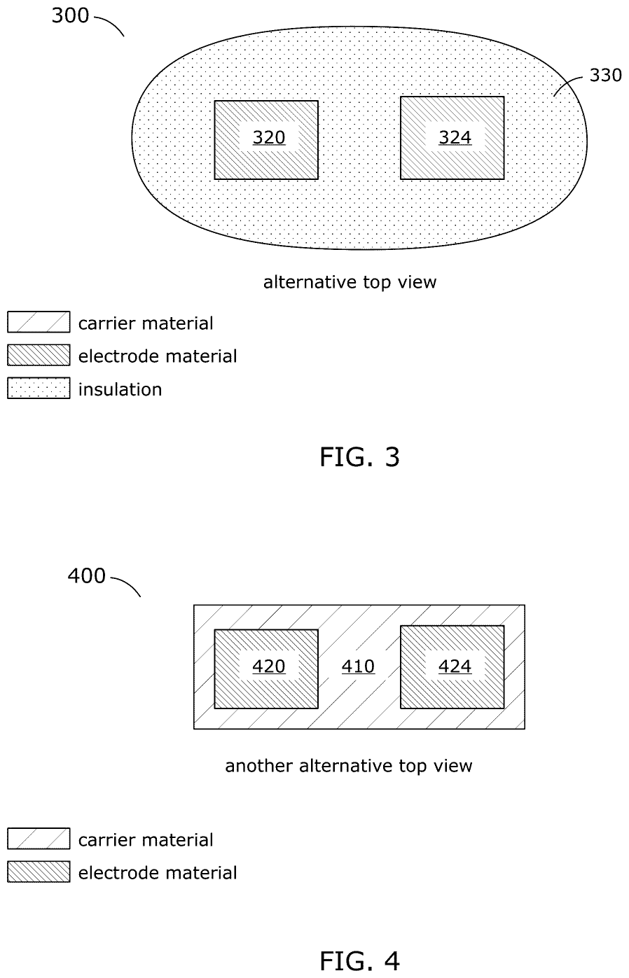

FIG. 3 illustrates an alternative top view 300 of a passive implantable device in an implementation of the disclosed technology. The device comprises a carrier (not visible), a first signal electrode 320, a first body potential electrode 324, and an insulating extension 330. The alternative implementation in FIG. 3 shows insulating extension 330 surrounding the perimeter of the carrier, and also covering its top. However, it exposes first signal electrode 320 and first body potential electrode 324. Similarly, insulating extension 330 may cover the bottom of the carrier, leaving a second signal electrode and a second body potential electrode exposed. In further implementations, insulating extension 330 may partially cover the top and/or bottom of the carrier, leaving the electrodes and other parts exposed.

FIG. 4 shows a top view 400 of an implementation without the optional insulating extension. The view of this implementation includes carrier 410, first signal electrode 420, and first body potential electrode 424.

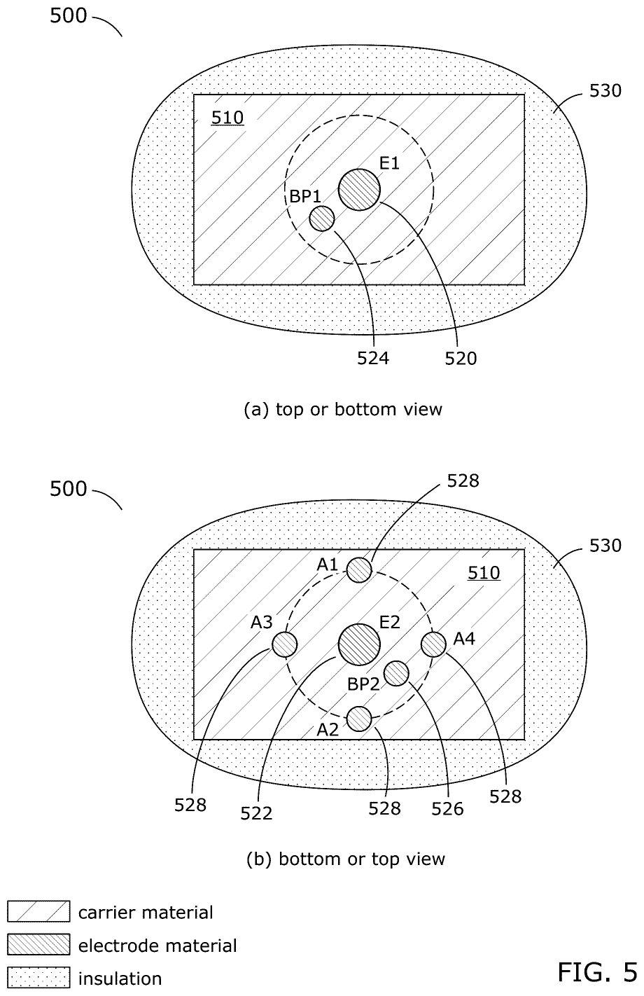

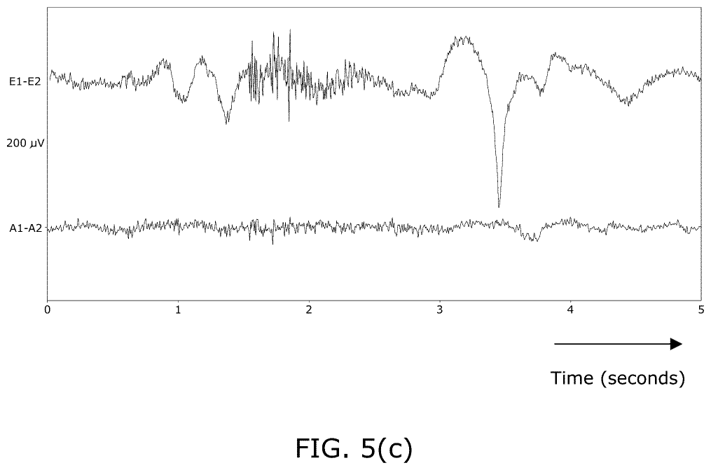

FIGS. 5(a) and (b) each illustrate an opposing view (top or bottom) of a device 500 in an implementation of the disclosed technology that is configured for measuring and/or compensating tangential signals. Tangential signals may be caused, for instance, by muscular activity. In a subgaleal position, signals related to muscular activity have a strong lateral component, whereas signals related to brain activity have a strong radial component. By measuring lateral and radial voltages separately, an implementation can reduce interference where one of the two is unwanted, or can measure both where both are signals of interest. FIG. 5(a) shows a view of the first side of the device, which may be passive or active. The first side includes carrier 510, first signal electrode 520 (E1), first body potential electrode 524 (BP1), and the optional insulating extension 530. Although first signal electrode 520 is shown in the center of carrier 510, and first body potential electrode 524 off-center, in different implementations their locations may vary and be anywhere else on carrier 510.

FIG. 5(b) shows a view of the second side of the device. It shows second signal electrode 522 (E2), which is opposite of and aligned with first signal electrode 520, and second body potential electrode 526 (BP2), which is opposite of and aligned with first body potential electrode 524. Around second signal electrode 522 are two or more pairs of lateral-signal electrodes 528 for measuring lateral signals. Two pairs are sufficient for measuring the amplitude and direction of any lateral signal. It is convenient for processing if two pairs are located orthogonally on a circle with second signal electrode 522 E2 in the center. However, in some implementations there may be more than two pairs, and the lateral-signal electrodes do not need to have regular positions visa vis each other. In further implementations, they may not be paired, but they could make up an array of electrodes. In yet other implementations, there may be just three lateral-signal electrodes, with E2 placed somewhere in the triangle in between.

In some implementations, the insulating extension 530 can reduce unwanted signal, for instance, caused by electrode-tissue displacement, propagating to the second signal electrode 522, lateral-signal electrodes 528 and the second body potential electrode 526.

FIG. 5(c) illustrates how the impact of electrode-tissue displacement caused by muscular activity, for example head movement in a sheep study, is reduced with the use of the insulating extension. In the sheep study, signal electrodes E2, A1-A2 were facing the skull, and signal electrode E1 was facing the scalp. Measurements show that the signal recorded by signal electrodes A1-A2 has less electrical noise caused by head movement compared with the signal recorded by signal electrodes E1-E2.

Nevertheless, as mentioned, having two pairs is convenient for signal processing, and FIG. 5(b) shows a first pair that includes electrodes A1-A2 and a second pair that includes electrodes A3-A4. The pairs are equidistant and positioned perpendicular to each other, with second signal electrode 522 E2 in the center.

While implanting, the subgaleal orientation matters. Obtaining best measurement depends on the position of nearby muscles. For example, if the device were implanted between the temporalis muscle and the skull, then lateral signals would be prevalent on the side of the muscle. The best measurement would be obtained with the first side oriented towards the brain and the second side oriented towards the muscle. In other situations, the best orientation may be otherwise.

To be robust against human error, an implementation may have lateral-signal electrodes 528 for measuring tangential signals on both sides of the device. Although this requires somewhat more electronics and might be slightly more expensive, such a device could be easier to use and could prevent inconvenience.

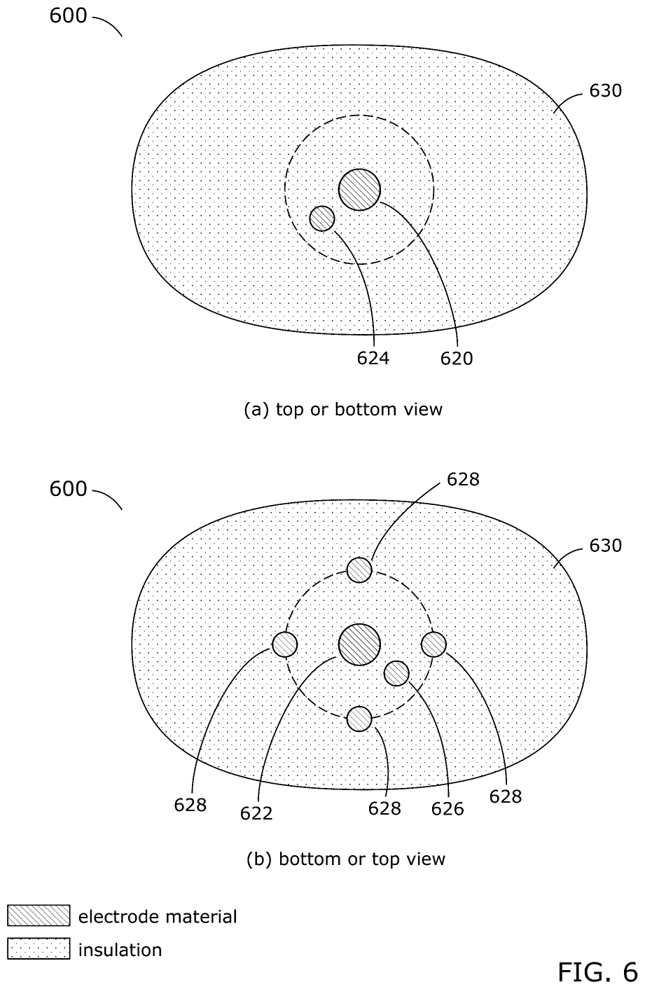

FIGS. 6(a) and (b) illustrate opposing views (top or bottom views) of a device 600 in an alternative implementation of the disclosed technology that is configured for measuring and/or compensating tangential signals. Device 600 includes the same elements as device 500, with like numbering. This alternative implementation is fully comparable with the implementation illustrated by FIGS. 5(a) and (b), but the insulation material of insulating extension 630 also covers the carrier other than at the locations of the electrodes.

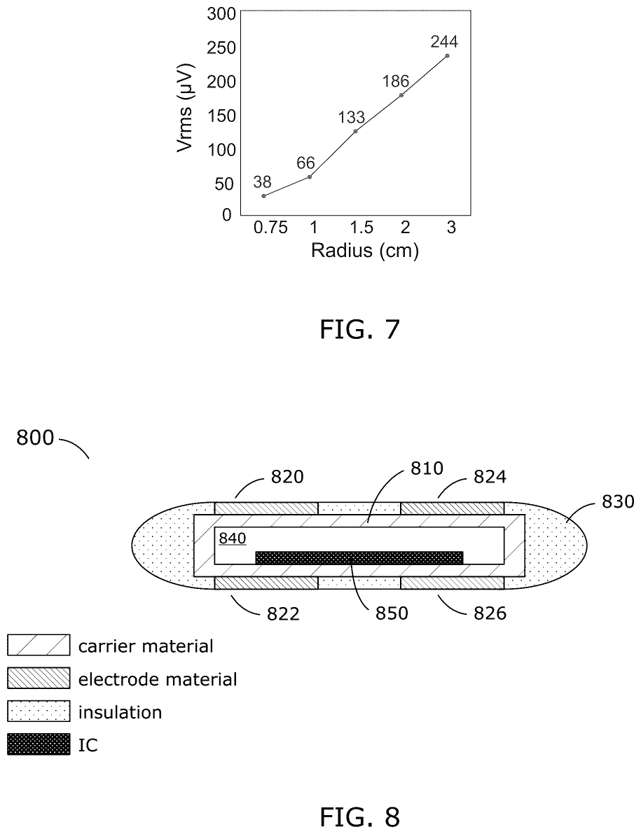

FIG. 7 shows in-vitro measurement results for different radii of the insulating extension in an implementation of the disclosed technology. In this measurement, a stimulus voltage applied on flesh tissue was measured with five passive devices whose insulating extension was circular. The devices only differed in the radius of the insulating extension. Measurement results show that the device's sensitivity is approximately proportional to the radius, providing evidence that implementations of the disclosed technology yield a strong improvement over traditional devices.

Active Devices

An active device is capable of more than just picking up measurement signals or delivering stimulation signals. For example, an active device that measures signals may provide local amplification, digitization, and storage of measured signals, and it may provide wireless (leadless) communication with an EIU. An active device may measure a differential signal that represents a channel of, for example, an electroencephalogram (EEG), an electrocardiogram (ECG), or an electromyogram (EMG). The active device functions require one or more electronic circuits that may be combined in an integrated circuit (IC). For the purpose of this document, the term IC is used to mean a monolithic semiconductor device, a combination of semiconductor devices and/or other electronic components mounted on a substrate or printed circuit board, as well as any thin film, thick film, or other printed electronics, and in general any combination of electronic components that provide the required functionality.

An active device is configured to transmit data to and/or receive data from the EIU. Data in this context may include information to control the active device or to communicate its status; measured signals and/or information about measured signals; stimulus signals and/or information about stimulus signals; firmware to operate inside the active device; and in general, any information required to support its functionality.

The electronic functions cost energy, which may be taken from a battery and/or other energy reservoir, such as a capacitor. For truly long-term operation, an energy reservoir must be rechargeable. Implementations charge the energy reservoir using harvested energy, or energy transferred by the EIU. Many methods of energy harvesting are known in the art, as well as methods of wirelessly charging a battery or other energy reservoir. For minimally invasive subgaleal operation, a device must be very thin. Even a tiny thin battery might store many days' worth of energy, potentially allowing a patient some retrieve from wearing the EIU. Convenient ways for transmitting energy from the EIU to the device include using an electric field, using an electromagnetic field, and using light.

To receive energy from an electric field, the device must include capacitor plates. The device already has potentially suitable capacitor plates in the form of a signal electrode and a body potential electrode. Implementations that use capacitive energy transfer separate energy transfer in time and/or frequency from signals measuring or delivering information. For example, electroencephalographic (EEG) signals range in frequency from below one Hertz to above 100 Hz. The implementation may receive capacitive energy from the EIU at, for example, a frequency of 10 MHz, leaving five orders of magnitude for a very effective frequency separation. Alternatively, or in addition, an implementation may receive energy in bursts of less than one millisecond, leaving ample time for undisturbed measurement of even the fastest EEG signals. Some implementations may separate signal electrodes and body potential electrodes from power transfer electrodes, not exposing power transfer electrodes to body tissues. Those implementations may cover power transfer electrodes with insulating material, for example material from the insulating extension. An implementation may also use capacitive coupling for communication between the device and the EIU. Again, capacitive plates for communication may be separate from or combined with power transfer electrodes, and/or signal electrodes and a body potential electrode. A simple implementation may have just four electrodes, two of which are short-circuited to each other to serve as body potential electrodes, and two of which serve as signal electrodes. When implanted between the scalp and the skull, one signal electrode and one body potential electrode face the scalp. These two electrodes can also serve as power transfer electrodes and as communication electrodes. Sensing or stimulating, power transfer, and communication may all be separate from each other in frequency, and in time. Additionally, communication itself may be bidirectional using frequency division multiplexing and/or time division multiplexing.

To receive energy from an electromagnetic field, a device must include an inductor. Such an inductor may also be used for communication between the EIU and the device. Implementations that use an inductor for power transfer and/or communication may locate the inductor around the perimeter of the carrier, for example inside the insulating extension, so that the inductor does not add to the thickness of the device. A combination of energy transfer and data transfer may be achieved with backscattering techniques, such as used in a radio frequency identification (RFID) system. Alternatively, an implementation may simply separate the two functions in time, for example by transmitting energy for a part of every second, and transmitting data for the remainder of the second, and/or separate the two functions in frequency, allowing continuous power transfer and continuous communication. Again, communication itself may be bidirectional using frequency division multiplexing and/or time division multiplexing. One form of electromagnetic coupling—resonant inductive coupling—uses inductor-capacitor pairs tuned to a specific frequency. Some implementations may use resonant inductive coupling, whereas other implementations may use simple inductive coupling to transfer energy and/or data electromagnetically. For the purpose of this document, inductive coupling or transfer and electromagnetic coupling or transfer are deemed the same. Resonant-inductive coupling forms a subset defined by the use of a tuned inductor to create a pole or zero in the transfer function at the frequency used for the coupling.

To receive light energy, the device must include a photovoltaic cell or other optical power transducer. The optical power transducer may conveniently sit inside the carrier if the carrier is made of a transparent material such as diamond. The EIU may transmit light through the skin into a cavity in the carrier, thereby providing power to the device. An implementation may also use light to transmit data from the EIU to the device. The data can be superimposed on the power. This approach is most suitable for implementations that stimulate and need only one-way transfer of data. For bidirectional optical communication, the device needs to include a light source, such as a light emitting diode (LED). Alternatively, a device may communicate optically in one direction, and inductively or capacitively in another direction.

Generally, to enable efficient transfer of power and data, an implanted active device must be aligned with the EIU. As is known in the art, this may be easily achieved with magnets. One or more magnets placed in the active device, for example in the insulating extension, may magnetically match with (and attract) one or more magnets placed in the EIU, ensuring correct positioning of the EIU versus the active device.

FIG. 8 illustrates a sectional view of an example active implantable device 800 with capacitive power transfer and communication in an implementation of the disclosed technology. Active implantable device 800 includes carrier 810, first signal electrode 820, second signal electrode 822, first body potential electrode 824, second body potential electrode 826, and optional insulating extension 830. Carrier 810 has a cavity 840, in which IC 850 is located. The drawing does not show any of the vias (pass-throughs) in the carrier that are used to electrically couple the electrodes, and/or other parts that are external to the carrier, with IC 850. In the implementation shown, the electrodes (first signal electrode 820, second signal electrode 822, first body potential electrode 824, and second body potential electrode 826) are deposited on the carrier, for instance by using CVD on a diamond carrier material, and depositing a film of doped electrode material. For example, if the electrode material is doped with nitride, it may be an n-type doped semiconductor material; or if it is doped with boron, it may be a p-type semiconductor material. In an alternative implementation, carrier 810 may comprise an organic material such as epoxy onto which platinum electrodes have been sputtered or otherwise deposited. The first body potential electrode 824 is electrically coupled (short-circuited) to second body potential electrode 826. All electrodes are electrically coupled with IC 850. Cavity 840 may further include a battery or other energy reservoir (not drawn). An EIU may be located in close proximity to active implantable device 800, with mostly skin tissue in between. The EIU may include capacitive plates whose size and positioning match those of first signal electrode 820 and first body potential electrode 824. The EIU powers active implantable device 800 by applying a high-frequency voltage to its capacitive plates. The voltage creates a first electric field between a first EIU plate and first signal electrode 820 and a second electric field of the same strength between first body potential electrode 824 and a second EIU plate, provided that IC 850 shows a low impedance at the field frequency between first signal electrode 820 and first body potential electrode 824. The electric field and the IC's low impedance path cause an alternating current to flow through active implantable device 800, which it uses to harvest energy that it may store in the energy reservoir. The implementation may interrupt the energy flow for communication with the EIU, or it may communicate with the EIU at a frequency substantially different from the energy transfer frequency to allow for communication and energy transfer to occur simultaneously.

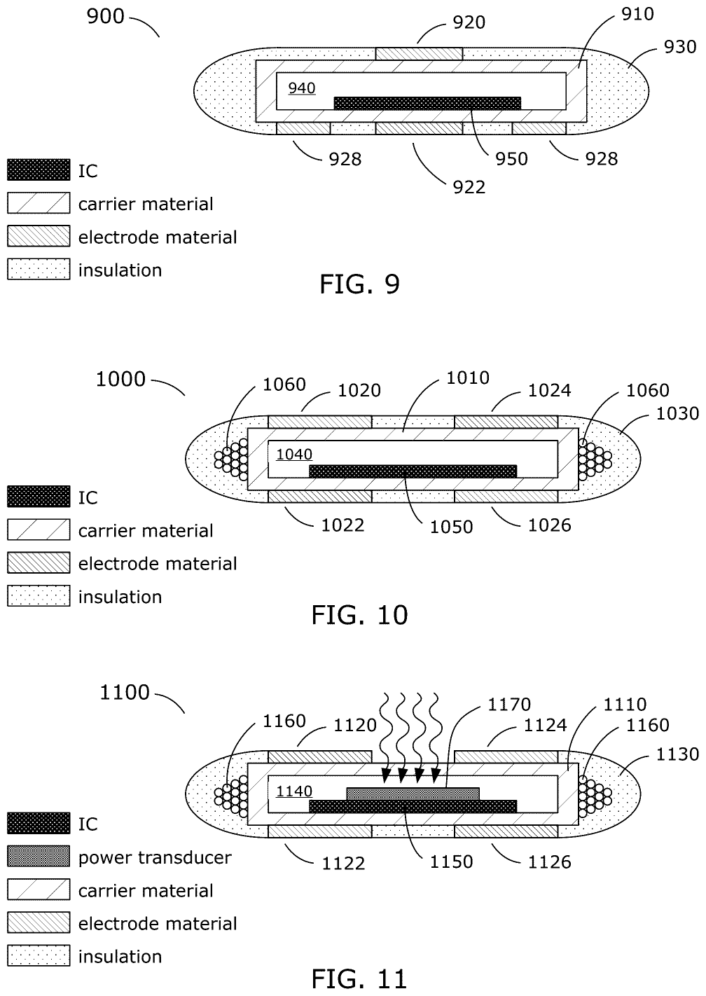

FIG. 9 illustrates a sectional view of an example active implantable device 900 with electrodes as shown in FIG. 6. Active implantable device 900 includes carrier 910, first signal electrode 920, second signal electrode 922, two or more instances of lateral-signal electrode 928, and insulating extension 930. Carrier 910 has cavity 940, in which IC 950 is mounted. This sectional view does not show the first and second body potential electrodes. The drawing also doesn't show any of the vias (pass-throughs) in the carrier that are used to electrically couple the electrodes, and/or other parts that are external to the carrier, with IC 950. Active implantable device 900 may receive energy and communicate capacitively on the second side, i.e. the side with second signal electrode 922 and the instances of lateral-signal electrode 928. Alternatively, the device could have communication and energy transfer electrodes on either side. Yet alternatively, the device may communicate and/or receive energy inductively or electromagnetically using an inductor inside insulating extension 930.

FIG. 10 illustrates a sectional view of an example active implantable device 1000 with inductive or electromagnetic power transfer and communication in an implementation of the disclosed technology. Active implantable device 1000 includes carrier 1010 with first signal electrode 1020, second signal electrode 1022, first body potential electrode 1024, second body potential electrode 1026, insulating extension 1030 (which is optional), and inductor coil 1060. The drawing does not show any of the vias (pass-throughs) in the carrier that are used to electrically couple the electrodes, inductor, and/or other parts that are external to the carrier, with IC 1050. Carrier 110 has a cavity 1040, in which IC 1050 is located. First body potential electrode 1024 is electrically coupled (short-circuited) to second body potential electrode 1026. First signal electrode 1020, second signal electrode 1022, first body potential electrode 1024, second body potential electrode 1026, and inductor coil 1060 are all electrically coupled with IC 1050. Inductor coil 1060 is configured to receive electromagnetic power and data from an EIU (not shown). In some implementations it is also configured to transmit data to the EIU. Inductor coil 1060 may be tuned to a specific frequency for resonant-inductive coupling, for example by a parallel or series capacitor, in which case energy transfer and communication must both occur at that specific frequency. In some implementations, inductor coil 1060 is not tuned to any specific frequency, and energy transfer and communication may take place at separate frequencies.

FIG. 11 illustrates a sectional view of an example active implantable device 1100 with optical power transfer and inductive or electromagnetic communication in an implementation of the disclosed technology. Active implantable device 1100 includes carrier 1110 with first signal electrode 1120, second signal electrode 1122, first body potential electrode 1124, second body potential electrode 1126, and insulating extension 1130 (optional) with inductor 1160. Carrier 1110 has a cavity 1140, in which are located IC 1150 and power transducer 1170. The drawing does not show any of the vias (pass-throughs) in the carrier that are used to electrically couple the electrodes, inductor, and/or other parts that are external to the carrier, with IC 1150. Power transducer 1170 may be, or include, a photovoltaic cell or other transducer that converts optical energy into electrical energy. Power transducer 1170 may be placed on top of IC 1150, as drawn, or may be placed in a different arrangement. An EIU or other source may shine light (visible or not visible) through a transparent side of carrier 1110 to illuminate power transducer 1170. For example, if carrier 1110 comprises polycrystalline diamond, it may be transparent and light energy can reach power transducer 1170 unhindered. As discussed previously, power transducer 1170 can also function to receive data from the EIU. If active implantable device 1100 also comprises a light source such as a LED, it may also transmit data towards the EIU. In many practical implementations, inductor 1160 will handle communication both from the EIU to active implantable device 1100 and back.

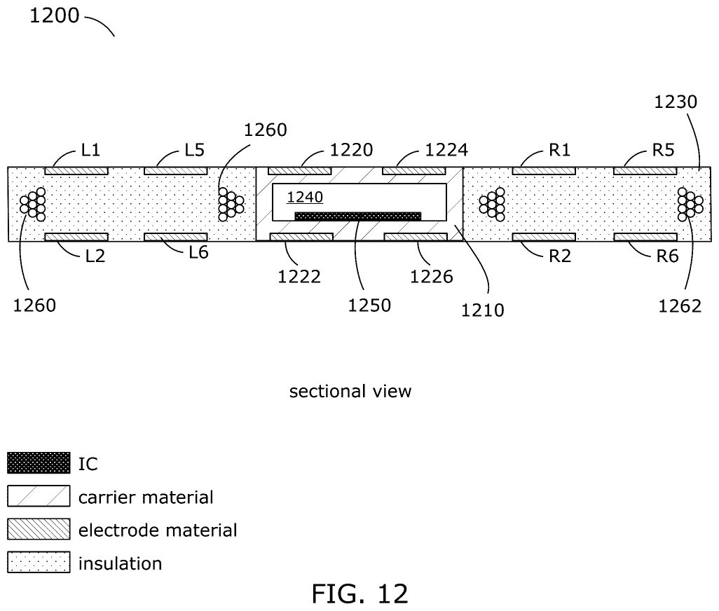

FIG. 12 illustrates a sectional view of an example active implantable device 1200 in an implementation of the disclosed technology. Active implantable device 1200 includes a carrier 1210 with first signal electrode 1220, second signal electrode 1222, first body potential electrode 1224, second body potential electrode 1226, first inductor 1260, second inductor 1262, signal electrodes L1-L8, signal electrodes R1-R8, and insulating extension 1230. The carrier has a cavity 1240, in which IC 1250 is located. The drawing does not show any of the vias (pass-throughs) in the carrier that are used to electrically couple the electrodes, inductor, and/or other parts that are external to the carrier. This example implementation exposes the carrier. However, other implementations may surround all parts of the carrier with either insulation or electrode material.

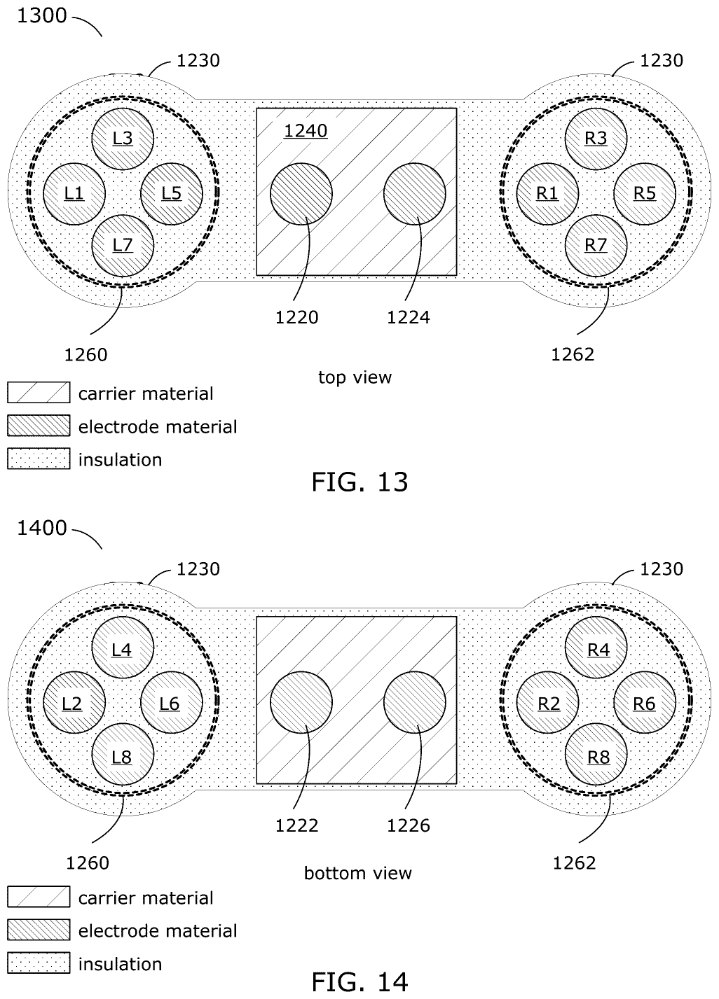

FIG. 13 illustrates a top view 1300 of the implantable device 1200 of FIG. 12 in an implementation of the disclosed technology. Although top view 1300 shows first inductor 1260 and second inductor 1262, those are inside insulating extension 1230 and may not be visible from the outside.

FIG. 14 illustrates a bottom view 1400 of the implantable device 1200 of FIG. 12 in an implementation of the disclosed technology. Although bottom view 1400 shows first inductor 1260 and second inductor 1262, those are inside insulating extension 1230 and may not be visible from the outside.

Configurable Devices

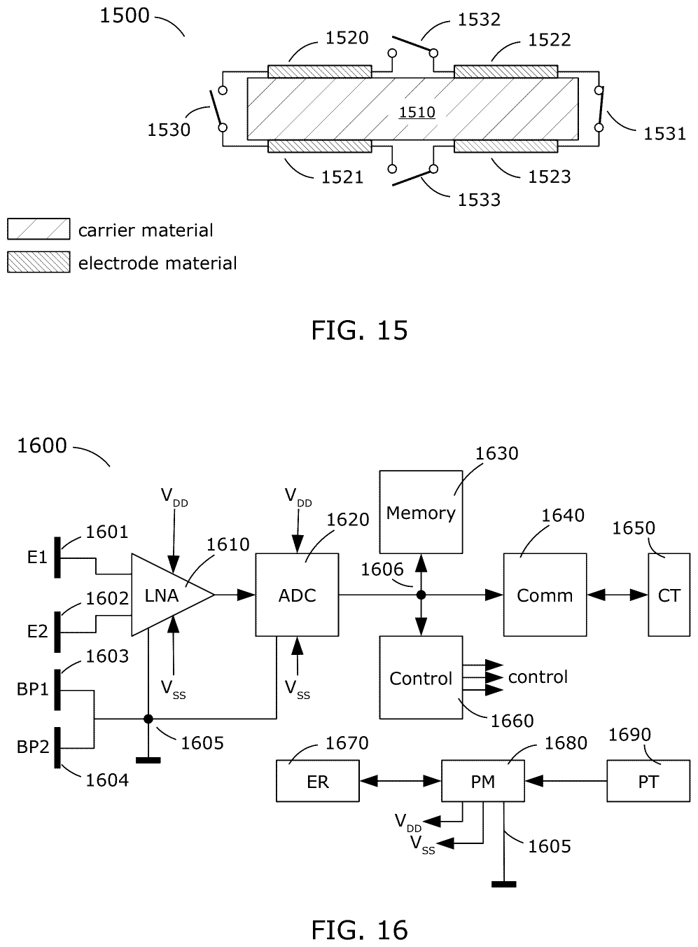

FIG. 15 illustrates an implantable device 1500 with configurable body potential electrodes in an implementation of the disclosed technology. Implantable device 1500 includes carrier 1510, which may be solid or hollow, and it may be made from a single piece of material or from two or more components sealed together. Carrier 1510, made of a first biocompatible electrically insulating material, has a first side and a second side, drawn here as its top and its bottom, that are on different, for example opposing, surfaces. In some implementations the first side and the second side may not be flat, but each may occupy multiple surfaces, and surfaces from the first side are offset from surfaces on the second side. The first side comprises electrode 1520 and electrode 1522, whereas the second side comprises electrode 1521 and electrode 1523. Each of the four electrodes may be configured to act as a signal electrode or a body potential electrode. The implementation comprises switch 1530 and switch 1531, and may comprise further switches, for example switch 1532 and switch 1533. As drawn, switch 1531 is closed and the other switches are open, which functionally renders electrode 1522 and electrode 1523 into body potential electrodes, and electrode 1520 and electrode 1521 into signal electrodes. Conversely, if switch 1530 is closed and switch 1531 is open, electrode 1520 and electrode 1521 are configured as body potential electrodes, and electrode 1522 and electrode 1523 as signal electrodes. The switches may be mechanical, semiconductor based, fusible, or programmable or settable in any other way. Any combination of switches is possible and within the ambit of the presently disclosed technology. Implantable device 1500 may be passive or active, and may function to sense electrical signals within tissue or to electrically stimulate tissue. It may include any of the features and functions described elsewhere in this document. FIG. 15 shows example switches that allow configuring each of the electrodes as either signal electrode or body potential electrode. However, implementations may comprise additional switches to electrically couple body potential electrodes to a ground node or common-mode node, and to electrically couple signal electrodes with any relevant amplifiers.

Architecture and Use Model

FIG. 16 illustrates an example functional architecture 1600 of an active sensing device in an implementation of the disclosed technology. The circuits of functional architecture 1600 may be incorporated on an IC that is mounted inside a device carrier. Functional architecture 1600 includes amplifier 1610, which may comprise a low-noise amplifier (LNA) or an instrumentation amplifier, analog-to-digital converter ADC 1620, memory 1630, which may include a non-transitory memory, communication unit 1640, communication transducer 1650, control unit 1660, energy reservoir 1670, which may comprise a battery and/or capacitor, power management unit 1680, and power transducer 1690. The electrodes first signal electrode 1601 (E1) and second signal electrode 1602 (E2) are coupled with differential signal inputs of amplifier 1610, whereas first body potential electrode 1603 (BP1) and second body potential electrode 1604 (BP2) are short-circuited to each other at node 1605, which is also coupled with a common-mode or virtual-ground input of amplifier 1610 and ADC 1620. Amplifier 1610 amplifies a differential input signal from first signal electrode 1601 and second signal electrode 1602, and provides the amplified signal to an analog signal input of ADC 1620 for digitization. ADC 1620 delivers the digitized signal to data bus 1606, which is also coupled with memory 1630, communication unit 1640, and control unit 1660. Control unit 1660 controls all functionality in functional architecture 1600, for example by executing logic instructions contained in firmware in memory 1630. For example, control unit 1660 can make functional architecture 1600 forward the digitized signals to memory 1630 for later communication to an EIU, or it can make functional architecture 1600 forward the digitized signals to communication unit 1640 for immediate communication to the EIU via communication transducer 1650. It may also include security functions to encrypt and protect transmitted signals, to authenticate an EIU, and to prevent communication with unauthorized external agents. Communication transducer 1650, as discussed earlier in this document, may comprise part or all of the electrodes E1, E2, BP1, and BP2; it may comprise other electrodes; it may comprise an inductor; it may comprise a photosensor and/or actuators, including photodiodes, photovoltaic diodes, and LEDS; or it may comprise any combination of these. Control unit 1660 may, via data bus 1606, communication unit 1640 and communication transducer 1650, communicate with the EIU to receive control information, firmware updates, etc., or to provide status information. Power management unit 1680 is electrically coupled with first body potential electrode 1603 and second body potential electrode 1604 via node 1605, and it is electrically coupled with power transducer 1690. Power transducer 1690 is configured to receive energy from the EIU to power the IC. Power management unit 1680 receives energy from power transducer 1690 and delivers power supply voltages VDD and VSS, which may be positive and negative, respectively, versus the body potential at node 1605. Power management unit 1680 manages energy reservoir 1670, for example by charging it when power transducer 1690 delivers more energy than needed for immediate operations, or depleting it when power transducer 1690 delivers insufficient energy for immediate operations. In some implementations, power management unit 1680 may include a body referencing circuit (BRC) that uses information from amplifier 1610 to determine an average voltage (common-mode voltage) between E1 and E2, and force this voltage on both the virtual ground at node 1605 and the body potential electrodes BP1 and BP2. The BRC may use negative feedback to generate a stable voltage halfway between average voltages of first signal electrode 1601 and second signal electrode 1602. It may then apply the stable voltage to first body potential electrode 1603 and second body potential electrode 1604. In implementations, power transducer 1690 may comprise part or all of the electrodes E1, E2, BP1, and BP2; it may comprise other electrodes; it may comprise an inductor; it may comprise a photovoltaic diode; or it may comprise any combination of these. In further implementations, power transducer 1690 may comprise communication transducer 1650, or vice-versa. Although FIG. 16 has been drawn as having fixed electrodes, E1, E2, BP1, and BP2 may be configurable as described with reference to FIG. 15.

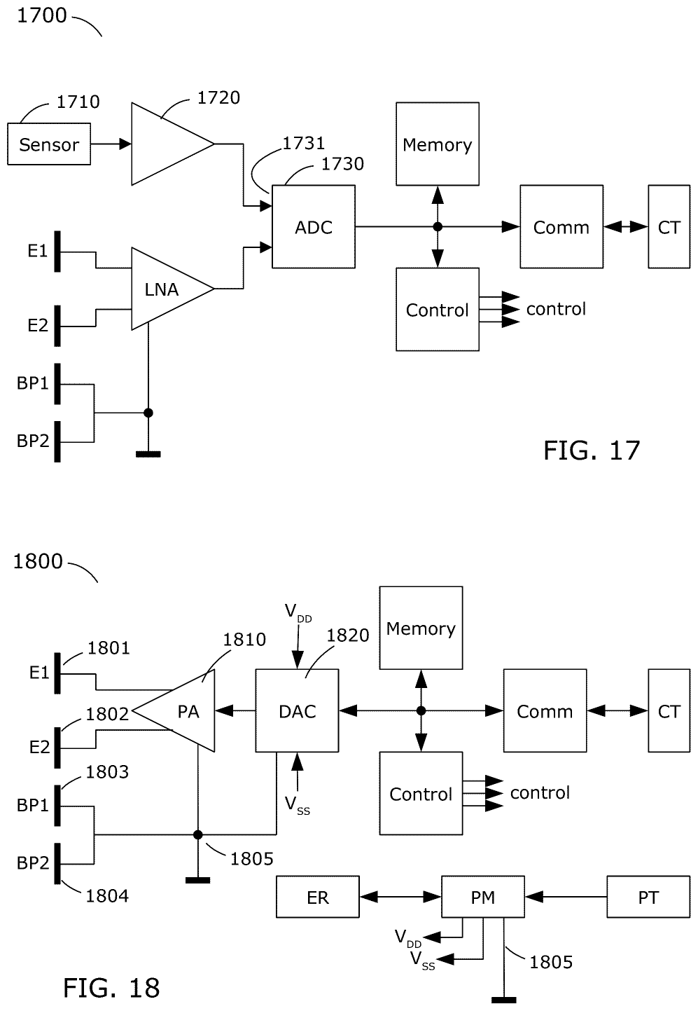

FIG. 17 illustrates an example functional architecture 1700pf an active sensing device with an extra sensor in an implementation of the disclosed technology. Functional architecture 1700 includes similar power management and supply features as functional architecture 1600, although they are not drawn here. The architecture supports an auxiliary sensor 1710 that may be mounted inside the active sensing device, or it may be placed externally. Sensor 1710 provides a measurement signal which is amplified by amplifier 1720. ADC 1730 receives the amplified measurement signal at an auxiliary input 1731, digitizes it, and passes it to the remainder of the system in the same fashion as measurement signals from electrodes E1 and E2 are passed on. Sensor 1710 may be, or include, any sensor that is relevant inside living tissue, including a sensor for temperature, blood flow, blood oxygenation, blood composition, blood pressure, heartbeat, etc. For example, sensor 1710 may include a photodiode mounted inside the device's carrier, receiving light that enters the carrier through its transparent bottom. Sensor 1710 could also include an LED illuminating tissue below the carrier, so that light reflected by the tissue and received by the photodiode may provide a measure for blood oxygenation. In another example, sensor 1710 may be, or include, a MEMS accelerometer mounted inside the device's carrier. Although the implementation in FIG. 17 shows functional architecture 1700 with one sensor amplifier and one LNA, and ADC 1730 with two inputs, other implementations may have additional sensors, amplifiers, and ADC inputs. Yet other implementations may combine amplifier 1720 with the LNA so that ADC 1730 does not need an extra input. However, those implementations may use a multiplexer at the amplifier/LNA input to switch between electrodes and any (other) sensors. Although FIG. 17 has been drawn as having fixed electrodes, E1, E2, BP1, and BP2 may be configurable as described with reference to FIG. 15.

FIG. 18 illustrates an example functional architecture 1800 of an active stimulating device in an implementation of the disclosed technology. The architecture is very similar to functional architecture 1600, with the biggest difference that instead of amplifier 1610 and ADC 1620 it comprises digital-to-analog converter DAC 1820 and power amplifier PA 1810. In contrast to FIG. 16, where the signal direction is generally from the left to the right, in FIG. 18 the signal direction is generally from the right to the left. In other words, data comes from the EIU (not drawn) via the communication transducer (CT) and the communication unit to DAC 1820, which converts it from the digital to the analog domain, and presents it to PA 1810, which amplifies the signal and presents it as a differential stimulus signal between first signal electrode 1801 and second signal electrode 1802, whose signals it generally balances symmetrically around the body potential 1805 measured at first body potential electrode 1803 and second body potential electrode 1804. In the implementation of FIG. 18, the power management unit is electrically coupled with first body potential electrode 1803 and second body potential electrode 1804 via body potential 1805, and it is electrically coupled with the power transducer. The power transducer is configured to receive energy from the EIU to power the IC. Although FIG. 18 has been drawn as having fixed electrodes, E1, E2, BP1, and BP2 may be configurable as described with reference to FIG. 15.

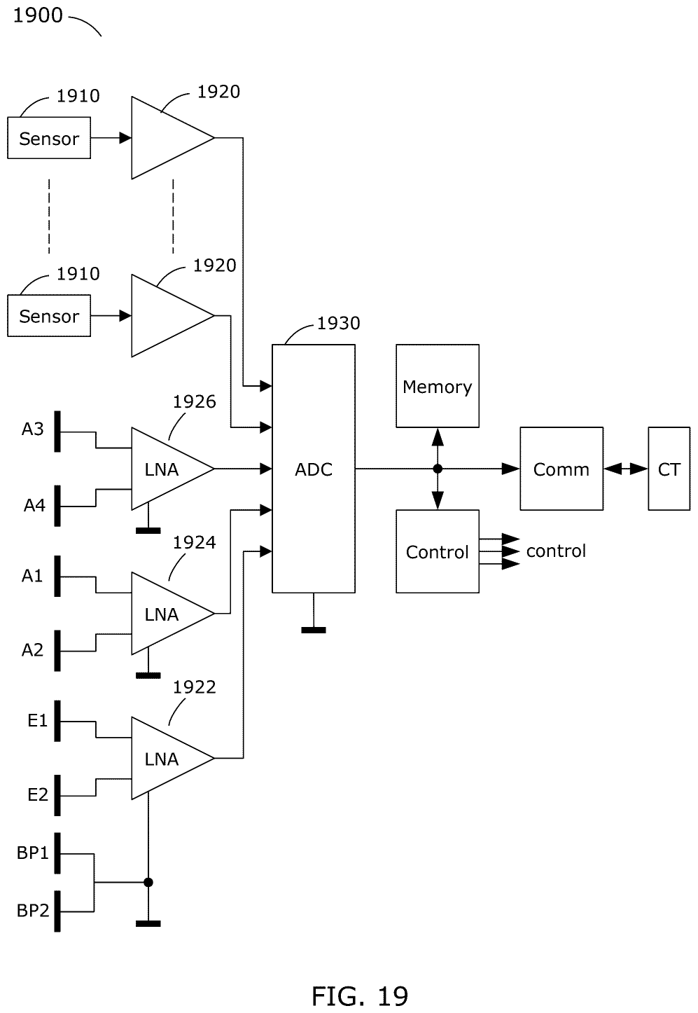

FIG. 19 illustrates an example functional architecture of an active sensing device 1900 with extra sensors and both radial and tangential sensing in an implementation of the disclosed technology. The architecture may be found in a device such as illustrated by FIGS. 5-6 that comprises two or more pairs of lateral-signal electrodes on the second side. The lateral-signal electrodes may be located on a circle centered around the second signal electrode, or they may be located in other positions. Sensing device 1900 includes one or more sensors 1910 (two have been drawn), that each may have an amplifier 1920. It further includes amplifier 1922, which may comprise an LNA or an instrumentation amplifier, to measure the signal between sensing electrodes E1 and E2, amplifier 1924, which may comprise an LNA or an instrumentation amplifier, to measure the signal between lateral sensing electrodes A1 and A2, and amplifier 1926, which may comprise an LNA or an instrumentation amplifier, to measure the signal between lateral sensing electrodes A3 and A4. Each of amplifier 1920 through amplifier 1926 may be coupled to a multiple-input ADC 1930 to digitize its input signal. In some implementations, there may not be a shared ADC, but multiple ADCs. In other implementations, amplifiers may be shared rather than be dedicated to a sensor or a pair of electrodes. In further implementations, there may be additional amplifiers for additional electrodes.

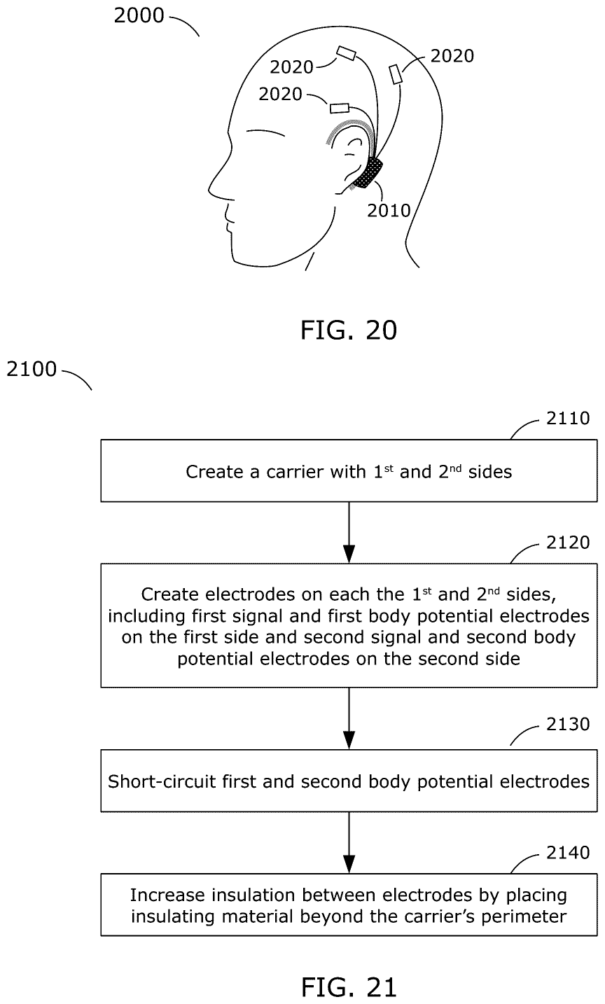

FIG. 20 illustrates an example external system 2000 in an implementation of the disclosed technology. External system 2000 includes the head of a patient or test subject, whose brain activity is monitored. The patient or test subject has one or more active devices subgaleally implanted below the scalp. Each active device communicates with an external interface unit EIU 2020, located on the scalp immediately over the respective implanted device. Each EIU 2020 is coupled with a signal processor 2010, for example using an external wire. The signal processor 2010 may be worn behind the ear, or hidden in an eyeglass frame, or it may be located in any other location that is not too inconvenient. Although FIG. 20 shows several ElUs placed over different lobes of the brain, an epilepsy monitor may function with as few as one device on each side of the brain. When necessary, or convenient, the external system 2000 may be removed. While the implanted devices store sufficient energy in their batteries or capacitors, monitoring may proceed, and the devices can store measured signals in internal memory.

Manufacturing

FIG. 21 illustrates a method 2100 of manufacturing an implantable device in an implementation of the disclosed technology. Method 2100 comprises the following steps.

Step 2110—Creating a carrier with a first side and a second side. The first side and the second side are on different surfaces, for example opposing surfaces, and the first side and the second side are less than 5 mm apart. The carrier may be solid or hollow, and it may be made from a single piece of material or from two or more components sealed together. It comprises a first biocompatible electrically insulating material. The material may comprise carbon, diamonds (monocrystalline, polycrystalline, microcrystalline and nanocrystalline), organics and polymers (such as SU8 or other types of epoxy, polyimide, different types of elastomer, different forms of parylene), and ceramics (aluminum oxide, aluminum nitride, glass, zirconium oxide, etc.), and any other biocompatible electrically insulating material.

Step 2120—Create electrodes on each the first side and the second side, wherein the first side has a first signal electrode and a first body potential electrode, and the second side has a second signal electrode and a second body potential electrode. Some implementations align the first signal electrode with the second signal electrode and align the first body potential electrode with the second body potential electrode. For aligning, the first signal electrode and the second signal electrode have equal areas, shapes, and orientations, and likewise the first body potential electrode and the second body potential electrode have equal areas, shapes, and orientations. The electrodes may comprise metal, such as platinum (in any of its forms), tungsten, titanium, gold, or any other metal that is suitable for implanting, or they may be carbon-based, such as diamond (e.g. boron-doped, nitrogen-doped, or otherwise doped, for any form of diamond, including monocrystalline, polycrystalline, microcrystalline and nanocrystalline), graphite, graphene, or any combination of diamond, graphite, and graphene, or they may comprise metal oxides and/or metal nitrites/nitrides (such as titanium nitride, indium tin oxide, iridium oxide), or any other electrically conductive materials suited for implantation. In an implementation, the electrodes are grown, deposited, implanted, or otherwise fabricated with any process steps known in the art, including sputtering, chemical vapor deposition (CVD), etc.

Step 2130—Creating electrical connections with at least part the electrodes, and electrically coupling the first body potential electrode to the second body potential electrode. An implementation may create the electrical connections at the same time and in the same manner as the electrodes, or it may create the electrical connections at a different time and in a different manner, for example by bonding an electrical wire to an electrode.

Step 2140—(Optional) increasing insulation between electrodes on the first side and electrodes on the second side by placing a biocompatible electrically insulating material beyond a perimeter of the carrier. The biocompatible electrically insulating material leaves the first signal electrode, the second signal electrode, the first body potential electrode, and the second body potential electrode exposed. The biocompatible electrically insulating material may comprise, for example, polydimethylsiloxane (PDMS, a polymer), or any other biocompatible material that is insulating and suitable for manufacturing.

### CONSIDERATIONS

Although the description has been described with respect to particular implementations thereof, these particular implementations are merely illustrative, and not restrictive. For example, elements described in one example implementation, but not in another one, may still be used or added to the other implementation. Although top and bottom views show an oval shape of the insulating extension, in implementations that insulating extension may be round, or have any other shape. And although most examples describe a limited number of signal electrodes and body potential electrodes, any number of electrodes may be used. Many variations of the architectures described in FIGS. 16-19 are possible, all achieving comparable results, and all are within the scope and ambit of this application. Although the EIU has been presented as a device that handles both energy transfer to the implanted device, and communication with the device, these functions may in some implementations be handled by separate external units.

Any suitable biocompatible materials may be used for the manufacture of implementations described herein. Positioning of elements may vary from those described herein. Conversion of signals between the analog and digital domains may occur at different positions in the architecture than described. Functions may be provided in software. Software and firmware may use any suitable programming language to implement the functionalities of particular implementations.

It will also be appreciated that one or more of the elements depicted in the drawings/figures can also be implemented in a more separated or integrated manner, or even removed or rendered as inoperable in certain cases, as is useful in accordance with a particular application.

As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise.

As used herein, the phrase one of should be interpreted to mean exactly one of the listed items. For example, the phrase “one of A, B, and C” should be interpreted to mean any of: only A, only B, or only C. As used herein, the phrases at least one of and one or more of should be interpreted to mean one or more items. For example, the phrase “at least one of A, B, or C” or the phrase “one or more of A, B, or C” should be interpreted to mean any combination of A, B, and/or C. The phrase “at least one of A, B, and C” means at least one of A and at least one of B and at least one of C.

Thus, while particular implementations have been described herein, latitudes of modification, various changes, and substitutions are intended in the foregoing disclosures, and it will be appreciated that in some instances some features of particular implementations will be employed without a corresponding use of other features without departing from the scope and spirit as set forth. Therefore, many modifications may be made to adapt a particular situation or material to the essential scope and spirit.

We claim:

1. An implantable device comprising:
a carrier with a first side and a second side, wherein the first side and the second side are on different surfaces that are less than 5 millimeters apart;
a first signal electrode, embedded on the first side;
a second signal electrode, embedded on the second side;
a first body potential electrode, embedded on the first side; and
a second body potential electrode, embedded on the second side, wherein the second body potential electrode is electrically coupled with the first body potential electrode.

2. The implantable device of claim 1 wherein:
the first signal electrode is aligned with the second signal electrode; and
the first body potential electrode is aligned with the second body potential electrode.

3. The implantable device of claim 1, further comprising:
three or more lateral-signal electrodes on the second side, located around the second signal electrode.

4. The implantable device of claim 1 wherein the carrier comprises at least one of:
diamond, ceramics, metal, or organics.

5. The implantable device of claim 1 further comprising an extension of insulating material beyond a perimeter of the carrier, the insulating material comprising a biocompatible electrically insulating material, and the extension exposing the first signal electrode, the second signal electrode, the first body potential electrode, and the second body potential electrode.

6. The implantable device of claim 1 comprising an integrated circuit (IC) inside the carrier, wherein:
the IC is electrically coupled with the first signal electrode, the second signal electrode, the first body potential electrode, and the second body potential electrode; and
the IC includes a power management system electrically coupled with the first body potential electrode, with the second body potential electrode, and with a power transducer configured to receive energy from an external interface unit (EIU) to power the IC.

7. The implantable device of claim 6 wherein the IC further comprises a memory configured for long-term recording of a digital signal.

8. The implantable device of claim 6 wherein the power transducer includes at least one of:
electrodes configured to receive power from the EIU through capacitive coupling;
an inductor coil configured to receive power inductively and/or electromagnetically; or
an optical power transducer configured to receive power optically, wherein the optical power transducer may be placed inside the carrier and may receive power transmitted by the EIU through the carrier.

9. The implantable device of claim 6 including a communication interface configured to transfer data between the IC and the EIU, wherein the communication interface comprises at least one of:
electrodes configured to communicate with electrodes in the EIU through capacitive coupling;
an inductor coil configured to communicate with the EIU through electromagnetic coupling;
a light source configured to send data optically, wherein the light source may be placed inside the carrier and may transmit data to the EIU through the carrier; or
an optical sensor configured to receive data optically, wherein the optical sensor may be placed inside the carrier and may receive data transmitted by the EIU through the carrier.

10. The implantable device of claim 9 wherein the IC is configured to provide a differential stimulus signal between the first signal electrode and the second signal electrode, wherein the differential stimulus signal has a common-mode component equal to a potential on the first body potential electrode and the second body potential electrode.

11. The implantable device of claim 6 wherein the IC is configured to sense a differential signal between the first signal electrode and the second signal electrode, wherein the differential signal has a common-mode component equal to a potential on the first body potential electrode and the second body potential electrode.

12. The implantable device of claim 11 further comprising a body referencing circuit (BRC) that generates a stable voltage halfway between average voltages of the first signal electrode and the second signal electrode, wherein the BRC applies the stable voltage to the first body potential electrode and the second body potential electrode.

13. The implantable device of claim 11 wherein the IC further comprises an amplifier (LNA) and an analog-to-digital converter (ADC), the LNA configured to amplify the differential signal, and the ADC configured to convert the amplified differential signal from an analog domain signal to a digital domain signal, the IC further comprising a circuit to communicate the digital domain signal to the EIU.

14. The implantable device of claim 13, further comprising a sensor coupled with an amplifier, wherein the amplifier is configured to amplify a sensor signal and to provide the amplified sensor signal to the ADC.

15. The implantable device of claim 14, wherein the sensor comprises an accelerometer.

16. The implantable device of claim 11 wherein the differential signal represents a channel of one of an electroencephalogram (EEG), an electrocardiogram (ECG), and an electromyogram (EMG).

17. The implantable device of claim 11, further comprising two or more pairs of lateral-signal electrodes on the second side, located on a circle centered around the second signal electrode, wherein:
each of the two or more pairs of lateral-signal electrodes is electrically coupled with an amplifier in the IC; and
the IC is configured to sense a lateral differential signal between each of the two or more pairs of lateral-signal electrodes.

18. The implantable device of claim 10 wherein the IC further comprises a power amplifier (PA) and a digital-to-analog converter (DAC), the DAC configured to convert a signal in a digital domain, received from the EIU via the communication interface, to an analog domain signal, and the PA configured to amplify the analog domain signal for presentation as a differential signal between the first signal electrode and the second signal electrode.

19. An implantable device with configurable electrodes comprising:
a carrier with a first side and a second side, wherein the first side and the second side are on different surfaces;
a first electrode and a second electrode located on the first side;
a third electrode and a fourth electrode located on the second side;
a first switch, electrically coupled with the first electrode and the third electrode, wherein the first switch configures the first electrode and the third electrode as signal electrodes when it is in an open position and as body potential electrodes when it is in a closed position; and
a second switch, electrically coupled with the second electrode and the fourth electrode, wherein the second switch configures the second electrode and the fourth electrode as signal electrodes when it is in an open position and as body potential electrodes when it is in a closed position.

20. A method to manufacture an implantable device comprising:
creating a carrier with a first side and a second side, wherein the first side and the second side are on different surfaces;
creating electrodes on each the first side and the second side, wherein the first side comprises a first signal electrode and a first body potential electrode, and the second side comprises a second signal electrode and a second body potential electrode; and
electrically coupling the first body potential electrode to the second body potential electrode.

21. The method of claim 20, further comprising:
using a biocompatible electrically insulating first material to create the carrier, wherein the first material comprises one or more of carbon, diamond, organics, a polymer, or a ceramic;
using a biocompatible electrically conducting second material to create an electrode, wherein the second material comprises one or more of metal, doped carbon, doped diamond, a metal oxide, or a metal nitride;
performing one of growing, implanting, and depositing to create an electrode; and
increasing insulation between an electrode on the first side and an electrode on the second side by placing a biocompatible electrically insulating third material beyond a perimeter of the carrier, the third material exposing the first signal electrode, the second signal electrode, the first body potential electrode, and the second body potential electrode, wherein the third material comprises one or more of metal, doped carbon, doped diamond, a metal oxide, or a metal nitride.
